(19) United States
(12) Patent Application Publication
(10) Pub. No.: US 2019/0239850 A1
(43) Pub. Date: Aug. 8, 2019

(54) AUGMENTED/MIXED REALITY SYSTEM AND METHOD FOR THE GUIDANCE OF A MEDICAL EXAM

(71) Applicants: Steven Philip Dalvin, Cambridge, MA (US); Matthew Sievers Alkaitis, New York City, NY (US)

(72) Inventors: Steven Philip Dalvin, Cambridge, MA (US); Matthew Sievers Alkaitis, New York City, NY (US)

(21) Appl. No.: 16/267,906

(22) Filed: Feb. 5, 2019

Related U.S. Application Data
(60) Provisional application No. 62/626,931, filed on Feb. 6, 2018.

(51) Int. Cl. A61B 8/00; G16H 10/20; G06T 19/00; G06T 7/70; A61B 90/00; G09B 5/06; G09B 19/00

(52) U.S. Cl. CPC A61B 8/42; G16H 10/20; G06T 19/006; G06T 7/13; A61B 90/36; G09B 5/06; G09B 19/00; G06T 7/70

(57) ABSTRACT

The present disclosure relates to a system and method capable of utilizing augmented reality and/or mixed reality to guide a user through the performance of a high-quality health-related examination. A head-mounted augmented reality device, smartphone, tablet or alternate display device allows the user to simultaneously view virtual exam guidance elements alongside real-world objects such as relevant anatomical landmarks or the exam detector. The virtual exam guidance elements are generated and/or updated in real-time based on pre-determined exam protocols as well as relevant data streams, such as data from cameras, sensors, the exam detector or other sources. The guidance elements are generated and/or positioned in 3D space in order to demonstrate to the user the preferred techniques or maneuvers that should be performed. In an exemplary embodiment, the system and method are designed for the performance of high-quality cardiac, venous, arterial, obstetric, genitourinary, abdominal or musculoskeletal ultrasound exams.

### CROSS-REFERENCE TO RELATED APPLICATIONS

The present application claims priority to U.S. Provisional Application No. 62/626,931, filed on Feb. 6, 2018 and titled, “AUGMENTED/MIXED REALITY SYSTEM AND METHOD FOR THE GUIDANCE OF A MEDICAL EXAM”, the disclosure of which is hereby incorporated by reference in its entirety.

### BACKGROUND OF THE DISCLOSURE

Augmented reality, commonly used interchangeably with mixed reality, augments an observer's view of the real world by displaying virtual visual information within the user's or the observer's view of the real world. This information can take many forms including but not limited to simple text, 3D virtual objects, imaging data, graphical labeling, and so on.

Incorporating virtual objects into an observer's view of the real world can be done through several different means such as through a projector system, a head-mounted-display (see-through or otherwise) where virtual objects are positioned within the user's direct line of sight to the real world, or through an electronics screen (i.e. computer screens, tablet screens, phone screens, etc) where the device's camera may serve as the observer's view of the real world and the screen is used to display virtual objects alongside real world objects. Examples of such devices may include the Microsoft Hololens (a see-through head-mounted display), the Magic Leap One headset (a see-through head-mounted display) and Apple's iPhone and iPad products, which may run software platforms such as AR-Kit to generate an augmented reality experience using a screen as a display.

Prior art discusses several instances in which augmented reality might provide benefit in the field of medicine, particularly with medical procedures. It has been proposed that augmented reality could be utilized to aid in localization of sites of interest noted on prior medical imaging, position a scalpel during a surgery, or even aid in the implantation of medical devices.

### BRIEF SUMMARY OF THE DISCLOSURE

The performance of high-quality medical exams requires the application of best-practice maneuvers and techniques. In real-world medical practice, however, patients may receive sub-optimal exams due to wide variation in the training, knowledge and capabilities of the medical provider. Many such exams require manual input, which makes it difficult to standardize exam protocols across the provider workforce. Failure to perform a high-quality exam may lead to incorrect or missed diagnoses, incorrect treatment plans, and ultimately patient harm. While there are many scenarios in which variation among clinicians' techniques and capabilities may lead to poor examination quality or medical error, one example that demonstrates these limitations particularly well is ultrasound imaging examinations. The quality of an ultrasound exam is particularly dependent on the skill and technique of the operator, and thus, ultrasound's clinical reliability is particularly susceptible to variations in clinician skill and ability. Accordingly, there is a need to improve the consistency of ultrasound exam quality and decrease variation among exams performed by different operators.

The present invention is a system and method for the guidance of health-related examinations. One exemplary set of components includes a display device, processor and detector capable of displaying digital information to the user, examinee or other observer via augmented reality and/or mixed reality in order to do one, two, or all of the following: improve the quality of the data acquired during the examination, reduce inter-operator variability, or enable a clinician to do a medical examination that was previously difficult or impossible to perform with good results due to limitations in skill or capability. The guiding elements may include but are not limited to text instructions, guiding graphics such as one or more arrows, targets, circles, color-changing elements, progress bars, transparent elements, angles, ‘ghost’ outlines of real-world objects, projections of real-time or stored imaging data, overlaid virtual organs or other virtual items. The guiding elements may exist and change according to a predetermined set of instructions, or in response to feedback elements such as movement as defined in a 3D coordinate system, time, acquired examination data, user input, observer input, examinee input, completion of exam instructions or a subset thereof, or lack of completion of exam instructions or a subset thereof. The guiding elements generated by the method may be updated before, during, or after the exam via any of these inputs alone or in combination. The disclosed method may generate or adapt visual or graphical elements in accordance with completion or lack of completion of the exam or a subset thereof. The disclosed method may display visual or graphical elements generated or updated according to exam instructions using augmented or mixed reality displayed with a projector system, a head-mounted-display (see-through or otherwise), or electronics screen (i.e. computer screens, tablet screens, phone screens, etc) such that the visual or graphical guide elements may be viewed simultaneously with the performance of the exam maneuvers.

In one exemplary embodiment of the disclosure, the method described above may be used to guide exam maneuvers related to appropriate placement or orientation of a diagnostic detector, such as an ultrasound probe. In such an embodiment, the disclosed method may additionally generate visual or digital guide elements to instruct the user, examinee or other observer to perform certain physical maneuvers before, during or after collection of data with the detector device.

This summary is a simplified and condensed description of the underlying conceptual framework, enabling technology, and possible embodiments, which are further explained in the Detailed Description below. This Summary is not intended to provide a comprehensive description of essential features of the invention, nor is it intended to define the scope of the claimed subject matter.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a diagram depicting one exemplary set of components capable of implementing the system and method. In this example, the exam device is an ultrasound detector with a hardwire connection to a tablet display device;

FIG. 1B is a diagram depicting a second exemplary set of components capable of implementing the system and method. In this example, the exam device is an ultrasound detector with a wireless connection to a see-through head mounted display device;

FIG. 1C is a block diagram depicting an exemplary set of functional components, including embodiments of processing environment components capable of implementing the technology;

FIG. 2 is a flow chart illustrating an embodiment of a method implemented by the system to generate and display virtual exam guidance elements based on external landmarks and optionally, real-time data collected from the exam detector;

FIG. 3 is a flow chart showing detail of step 300 from FIG. 2, in which the method uses computer vision techniques and/or spatial sensor inputs to identify relevant examinee position and anatomical landmarks;

FIG. 4A is a flow diagram showing detail of step 400 from FIG. 2, in which data collected from a detector is analyzed in real time and the analysis is used to update virtual exam guidance elements;

FIG. 4B is a flow chart showing detail of step 410 from FIG. 4A, in which necessary data inputs are collected and integrated;

FIG. 4C is a flow chart of showing detail of step 420 from FIG. 4A, in which exam image quality is assessed; and

FIG. 4D is a flow diagram showing detail of step 430 from FIG. 4A, in which the method provides system adjustments and/or updated guidance elements to improve image quality;

FIG. 4E is a flow diagram showing detail of step 440 from FIG. 4A, in which visualized anatomy is analyzed;

FIG. 4F is a flow diagram showing detail of step 450 from FIG. 4A, in which the method provides updated guidance elements to improve the view of relevant anatomy;

FIG. 4G is a flow diagram showing detail of step 460 from FIG. 4A, in which the method analyzes maneuvers or other data acquisition over time;

FIG. 4H is a flow diagram showing detail of step 470 from FIG. 4A, in which the method updates guidance elements to improve performance of relevant anatomy or other data acquisition over time;

FIG. 5A is an illustration of an embodiment of the system and method in which virtual guidance is provided via an electronic display device for the performance of a cardiac ultrasound exam (echocardiogram);

FIG. 5B is an illustration of an embodiment of the system and method in which virtual guidance is provided via a head mounted display device for the performance of a cardiac ultrasound exam (echocardiogram);

FIG. 6A is an illustration of an embodiment of the system and method in which virtual guidance is provided via an electronic display device for the performance of a lower extremity vascular exam;

FIG. 6B is an illustration of an embodiment of the system and method in which virtual guidance is provided via a head mounted display device for the performance of a lower extremity vascular exam;

FIG. 7 is an illustration of an embodiment of the digital or virtual guidance elements described in the disclosed method, where the guidance elements are designed to aid the user in performing a lower extremity vascular exam; and

FIG. 8 is an illustration of how the algorithm described in FIG. 5A-H analyzes current detector-generated images to stored images, extracts key features, and creates virtual exam guidance elements.

### DETAILED DESCRIPTION

The technology discussed herein comprises a system and method of guiding a health-related examination generally including digital information displayed to the user, examinee or other observer via augmented reality and/or mixed reality in order to do one, two, or all of: improving the quality or quantity of the data acquired during the examination, reducing inter-operator variability, and/or enabling a clinician to do a medical examination that was previously difficult or impossible to perform with good results due to limitations in skill or capability.

The system utilized herein includes an augmented reality display device such as a head mounted display (see-through or otherwise) or electronic display device, as well as zero, one, or a plurality of medical examination devices that include but are not limited to imaging tools such as an ultrasound probe, ultrasound transducer, or camera, listening devices such as a stethoscope, physiologic measurement devices such as EKG leads, nerve conduction measuring devices, blood pressure cuffs, pulse oximeters, temperature probes, etc. Such a system may also include zero, one, or more sensors that gather information about the exam being performed, where the information gathered from the sensors may be utilized to aid in the examination.

During an examination, the augmented reality device displays virtual guiding elements relevant to performing the examination. The guiding elements may or may not be updated throughout the examination based on a number of factors. The user would then utilize these guiding elements as an aid to perform his or her examination. Data from the medical examination, including imaging data, video, or other data derived from a sensor or detector may be stored and/or transmitted throughout the exam in order to improve the content or quality of the virtual exam guidance elements, save exam data for future reference, allow another individual to observe the exam or allow interpretation of exam data.

The method utilized herein includes generating virtual guiding elements to guide positioning, orientation and/or activation of examination tools, where the virtual guiding elements may include but are not limited to instruction in the form of plain text or otherwise, one or more arrows, targets, circles, color-changing elements, progress bars, transparent elements, angles, overlaid virtual organs, ‘ghost’ outlines of real-world objects, projections of real-time or stored imaging data, or other virtual items. Furthermore, these guiding elements may also serve to guide positioning and/or orientation of the user, examinee, or other observer.

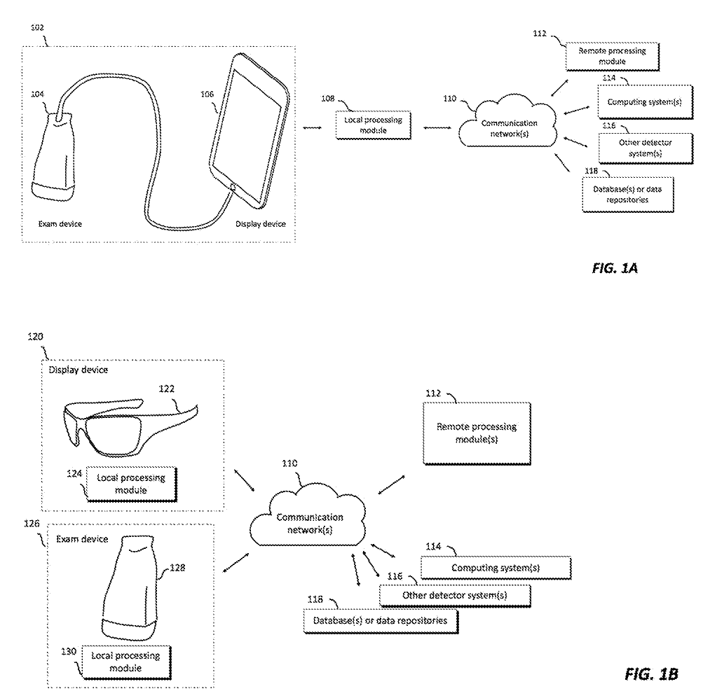

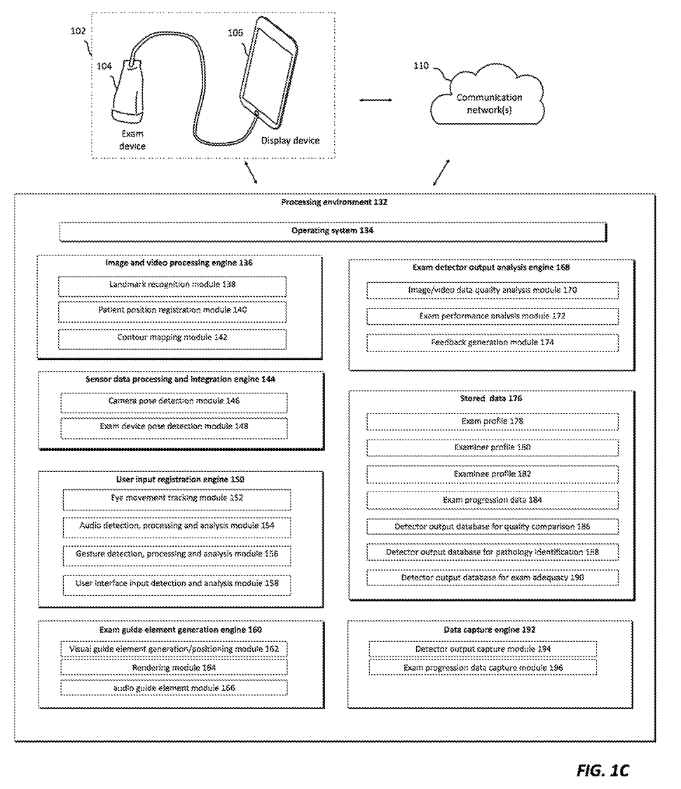

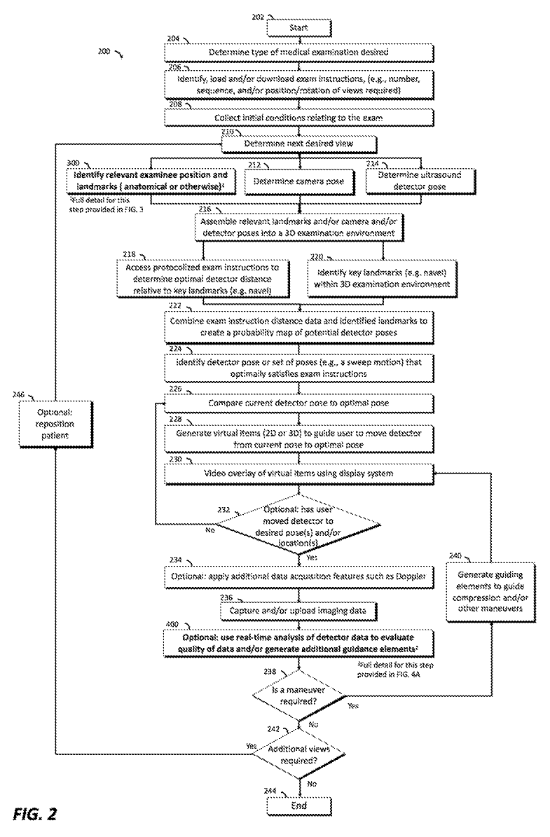

FIGS. 1A-C depict exemplary sets of hardware and functional components capable of implementing the system and method.

FIG. 1A is a diagram depicting one exemplary set of components capable of implementing the system and method. In this example embodiment, the exam device hardware system 102 is comprised of an ultrasound detector 104 with a hardwire connection to a tablet display device 106. To use this hardware system, the user would position the tablet display device 106 such that the devices camera would have an unobstructed view of the examinee. The user would view the examinee and the examination field on the device screen, where virtual guide elements could be displayed and perceived by the user. The virtual guide elements may appear as “2D” projections such that they appear to be in the plane of the device's screen, or they may appear as “3D” projections such that they appear to be positioned in 3D space alongside the real-world objects that are viewed through the device's camera and displayed on the screen. A computing system to manage the creation, positioning, modification and updating of the virtual guide elements may include a local processing module 108 which may use wifi, Bluetooth or other protocol to access a communication network 108 and interface with a remote processing module 112, other computing system(s) 114, other detector system (s) 116, or database(s) or data repositories 118.

FIG. 1B is a diagram depicting one exemplary set of components capable of implementing the system and method. In this embodiment, the display device 120 and exam device 126 are not hardwired together, but rather communicate independently with a communication network 110 via wifi, Bluetooth or other method. In this embodiment, the display device 120 is a head-mounted display device 122 capable of displaying virtual exam guidance elements juxtaposed with the user's direct line-of-sight view of the real world. In this embodiment, the exam device 126 is comprised of a wireless ultrasound probe 128. The display device 120 and exam device 126 may each have their own local processing modules 124 and 130, respectively.

FIG. 1C is a block diagram depicting an exemplary set of functional components, including embodiments of processing environment components capable of implementing an exam guidance service. The processing environment 132 may be supported and implemented by either local processing modules 108, 124, or 130 or by remote processing module(s) 112 connected to the hardware components 102 via a communication network 110, or by a combination of local and remote modules. As shown in the embodiment described in FIG. 1C, the processing environment 132 is comprised of an operating system 134, image and video processing engine 136, Sensor data processing and integration engine 144, User input registration engine 150, Exam guide element generation engine 160, Exam detector output analysis engine 168, Stored data 176 and Data capture engine 192.

The operating system 134 establishes the underlying software infrastructure that allows the hardware components of the processing module to run and interact with the functional software engines shown in FIG. 1C. The Image and video processing engine 136 allows image and video feeds to be interpreted into anatomical landmarks, patient position data and contour maps using the landmark recognition module 138, patient position registration module 140, and contour mapping module 142, respectively. Once patient positioning, anatomical landmarks and spatial contours are identified, these data may then be used as an input for the creation and display of virtual exam guidance elements.

The sensor data and integration engine 144 accesses sensor data that may include but is not limited to cameras, gyroscopes, accelerometers, depth-sensing systems and other sensors or systems, and uses these inputs to determine relative positional data. The camera pose detection module 146 determines the 3D spatial position or pose of the tablet display device, or, in other embodiments of the system, the pose of a head-mounted display device. The exam device pose detection module 148 determines the 3D spatial position or pose of the device used to obtain exam data, for example an ultrasound probe in certain embodiments. The positional and/or 3D spatial data in the camera pose detection module 146 and the exam pose detection module 148 may be derived using any number of techniques known to one skilled in the art including but not limited to visual odometry visual inertial odometry, and/or using other sensors and systems such as elements attached to the probe or any part thereof. Once the 3D spatial position of the camera or exam device is determined, this data may then be used as an input for the creation and display of virtual exam guidance elements.

The user input registration engine 150 integrates various user inputs to allow the user to interact directly with the system. In combination, various modules of the user input registration engine 150 allow the system to detect and interpret a range of user inputs to make selections, input needed data, modify the exam or other functions that require input from the user. The eye movement tracking module 152 tracks the user's eye movements relative to the head-mounted display device or other system component in order to determine the direction of the user's gaze, the point in space on which the user is focusing, speed of movement, or particular patterns of movement. The audio detection, processing and analysis module 154 detects audio inputs, such as spoken commands and performs appropriate analyses such as natural language processing to determine specific commands relevant to the performance, modification or conclusion of the examination. The Gesture detection, processing and analysis module 156 detects and interprets physical gestures by the user, such as hand gestures to indicate a selection from among a menu of options. The user interface input detection and analysis module 158 detects and interprets other types of manual inputs, such as pressing a button to change a detector setting or confirm the completion of a particular maneuver.

The Exam guide element generation engine 160 is a service that draws from multiple data sources in order to generate virtual exam guide elements that the user then perceives via the head-mounted display, screen or other display system. The visual guide element generation/positioning module 162 may utilize data such as information about the exam environment, the examinee's anatomical landmarks and other contour information generated by the image and video processing engines 136, camera or exam device 3D spatial positioning data from the sensor data processing and integration engine 144, various user inputs such as gaze direction, option selection or audio command from the user input registration engine 150, data derived from real-time analysis of the exam detector output generated by the exam detector and analysis engine 168, data such exam profile, user profile, examinee profile, progress data, or various output databases available as stored data 176, or other supplementary data. Using these data sources, the Exam guide element generation engine 160 is able to produce the design, 3D spatial positioning data, content, appearance and other features of virtual exam guide elements for the purpose of helping the user collect high-quality exam data, perform a specified physical maneuver, track the real-time quality and progress of the exam, or any other element of a high-quality exam. The rendering module 164 utilizes the data generated by module 162 and renders the virtual exam guide elements using a head-mounted augmented reality display system, smartphone or tablet display screen, or other display system such that the user may perceive the virtual exam guidance elements alongside real-world objects in the exam field and use these instructions to perform a high-quality exam. The audio guide element module 166 is able to use similar inputs as the visual guide element generation/positioning module 162 in order to create and play audio instructions to the user for a similar purpose of guiding the user to perform a high quality medical exam.

The virtual exam guide elements generated by engine 160 may include but are not limited to text instructions, guiding graphics such as one or more arrows, targets, circles, color-changing elements, progress bars, transparent elements, angles, ‘ghost’ outlines of real-world objects, projections of real-time or stored imaging data, overlaid virtual organs, or other virtual items. The guiding elements may exist and change according to a predetermined set of instructions, or in response to feedback elements such as movement as defined in a 3D coordinate system, time, acquired examination data, user input, observer input, examinee input, completion of exam instructions or a subset thereof, or lack of completion of exam instructions or a subset thereof. The guiding elements generated by the method may be updated before, during, or after the exam via any of these inputs alone or in combination. The disclosed method may generate or adapt visual or graphical elements in accordance with completion or lack of completion of the exam or a subset thereof. The disclosed method may display visual or graphical elements generated or updated according to exam instructions using augmented or mixed reality using a projector system, a head-mounted-display (see-through or otherwise), or electronics screen (i.e. computer screens, tablet screens, phone screens, etc) such that the visual or graphical guide elements may be viewed simultaneously with the performance of the exam maneuvers. These guiding elements may exist and change according to a predetermined set of instructions, or in response to feedback elements such as movement as defined in a 3D coordinate system, time, acquired examination data, user input, observer input, examinee input, completion of exam instructions or a subset thereof, or lack of completion of exam instructions or a subset thereof.

As noted, stored data 176 may be used as an input for other functional components of the system, and in particular the Exam guide element generation engine 160. Stored data may include, but is not limited to, exam profile data 178, user profile data 180, examinee profile 182, exam progression data 184, one or more detector output databases for quality comparison 186, one or more detector output databases for anatomy or anatomical pathology identification 186, one or more detector output databases for exam adequacy 190, or other stored data types or categories.

The Exam detector output analysis engine 168 allows real-time assessment of output from an exam detector, such as an ultrasound probe in one exemplary embodiment. The image/video data quality analysis module 170 determines whether the image or video data that is being generated by the exam detector is of sufficient quality to allow interpretation. The exam performance analysis module 172 determines whether the exam sequence, maneuvers and cumulative captured data are sufficient in combination to constitute a high-quality exam. The feedback generation module 174 allows the results of these analyses to be communicated to the exam guide element generation engine 160 in order to modify, update or re-design exam guidance elements.

The data capture engine 192 gathers data from the ongoing exam for the purposes of storing a record, building databases of stored data for future exams, communicating data to an interface for interpretation or other use. The detector output capture module 194 is responsible for capturing the output from the exam detector, such as an ultrasound probe in one exemplary embodiment. The exam progression data capture module 196 captures various data about the progress of the exam, such as the user's movements, the timing or features of virtual exam guidance elements, the video feed captured from a head-mounted display, smartphone or tablet device, the positional data generated by accelerometers, gyroscopes, depth sensing systems, or any other data pertaining to the progress of the exam.

FIG. 2 shows a flow chart illustrating an embodiment of a method implemented by the system to generate and display virtual exam guidance elements based on external landmarks and optionally, real-time data collected from the exam detector. This methodology may apply to any and all medical examinations in which virtual guiding elements are used to guide the examination.

Method 200 may be performed locally on an electronic device, online via a cloud system, or via some combination of the two. The method begins at 202. At 204, the type of medical examination desired is determined. Examples of medical examinations may include but are not limited to ultrasound examinations of the heart, gallbladder, abdominal aorta, carotid arteries, musculoskeletal structures, pregnant uterus, non-pregnant uterus, abdomen, lung, testicle, etc.

The type of medical examination may be determined simply by asking the user to input the desired exam, or it may automatically be determined through contextual information. This contextual information may include but is not limited to medical history information pertaining to one or more examinees, patient positioning, sensor data, visualized or detected body landmarks, or other health information.

At 206, the system identifies, loads, and/or downloads exam instructions that may be used to guide the performance of the examination. Exam instructions may include information such as the number of views required, the proper sequence in which the user might obtain the views, positional and/or rotational information pertaining to probe placement, and/or other instructive elements. At 208 data generally relating to the initial conditions of examination is optionally collected via sensors that may include but are not limited to images, video, one or more accelerometers, inertial data, and/or one or more gyroscopes. Examples of initial exam conditions may include but are not limited to user, examinee, or observer positioning, lighting, medical examination data, examinee medical information, or orientation of one or more involved devices, orientation of a user, examinee, or observer. While step 208 specifies initial conditions, and has a discrete location within the method, it should be noted that the system may collect data generally relating to examination conditions, initial or otherwise, at any time throughout the performance of method 200. At 210 the system may use the instructions from step 206, which may be modified by data gathered in step 208, to determine the next desired view for a given exam.

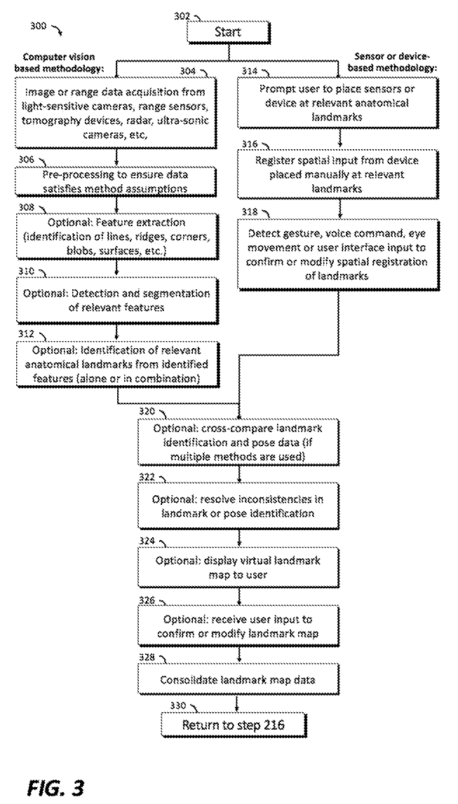

At 300, relevant examinee position and landmarks (anatomical or otherwise) are detected. These landmarks may include but are not limited to examination tools such as an ultrasound probe/transducer, stethoscope, audio capture device, video capture device, or temperature determining device, surfaces, body landmarks such as that of a user, examinee, and/or observer, and/or parts thereof including but not limited to parts of the medical examination devices and/or body parts such as hands, fingers, bone landmarks, etc. The detection of the landmarks may be through the use of sensor data. In one embodiment, the sensor is a camera and computer vision is utilized in order to determine body landmarks. In another embodiment, the user may manually choose landmarks with or without prompting. For example, the user may convey these landmarks by placing a device in the location of a landmark, by indicating the landmark with hand gestures, by indicating the landmark by selecting it on an electronic display device, or via one or more combinations of the aforementioned methods. Examinee position may be defined differently depending upon the exam. Some examples of relevant examinee positioning may include resting an examinee supine during an echocardiogram or lung pleura examination, or positioning an examinee's leg so as to expose the inguinal region to examine the femoral vein or artery. Anatomical landmarks may include any relevant anatomy pertaining to performing an exam. Examples of anatomical landmarks may include but are not limited to the popliteal fossa in a lower extremity vascular examination, the navel in an abdominal aortic aneurysm screen, or xyphoid process in an echocardiogram. Additional information detailing the methods through which step 300 is performed are provided in FIG. 3.

At 212, the system determines the camera pose, and at 214 the system determines the ultrasound detector pose within the 3D spatial environment. This is done using the sensor and integration engine 144 (described in FIG. 1C) to access sensor data such as cameras, gyroscopes, accelerometers, and/or other sensors or systems. Step 212 utilizes the camera pose detection module 146 (described in FIG. 1C) while step 214 utilizes the exam pose detection module 148 (described in FIG. 1C). As discussed in the detailed description of FIG. 1C, these modules, and therefore steps 212 and 214, determine 3D spatial and/or positional data using techniques that may include but are not limited to visual odometry, visual inertial odometry, and/or any number of techniques known to one skilled in the art.

At 216, the system assembles information from steps 300, 212, and 214, including relevant anatomical landmarks, camera pose information, and/or detector pose information, into a 3D examination environment. At 218, the system accesses the protocolized exam instructions from step 206 to determine optimal detector locations and/or distances relative to key landmarks. For example, the system may access the exam instructions and extract data indicating that an ultrasound detector should be placed a certain distance superior to the navel to begin an abdominal aortic aneurysm screening. At 220, relevant landmarks within the 3D examination environment are identified, and at 222, the information from steps 218 and 220 are combined in order to create a probability map of potential desired detector poses. For example, at step 222 the system may combine information from step 218 pertaining to the optimal distance from the navel that an ultrasound detector should be placed with information from step 220 pertaining to the locations of both the navel and the 12^{th} rib of an examinee within the context of the 3D examination environment. Using this information, the system may then create a probability map of potential optimal detector poses for the performance of an abdominal aortic aneurysm screening.

In this context, a probability map refers to a 3D spatial mapping of possible optimal detector poses, each weighted by the probability that a given pose will result in acquisition of the desired data. For example, step 218 may indicate that an examinee with a navel surrounded by convex body tissue (i.e. obese) may tend to have a poorly defined 12^{th} rib feature. Thus, when step 222 combines information from steps 218 and 220, it may choose to assign a lower probability to any detector poses derived using the examinee's 12^{th} rib feature. Once a probability map of possible detector poses has been created, the system identifies the pose or set of poses at step 224 to optimally satisfy the exam instructions. In the event that a set of poses is desired, step 224 may identify an area (rather than a single point) through which the ultrasound detector should pass to acquire the desired data (e.g. a “sweep” motion). For example, In the event that a single optimal detector pose is difficult to identify, a “sweep” motion allows the system to collect additional data and increase the probability that the desired examination data will be captured during at least one of the many poses that constitute a “sweep” when performed in sequence.

At 226, the system compares the current detector pose with the optimal pose or poses, and at 228 uses this comparison to generate virtual items (2D or 3D) for the purpose of guiding the user to move the detector from the detector's current pose to the optimal pose. The generation of virtual guiding elements is done in a similar manner to that described in FIG. 1A-1C. As described above, virtual guiding elements can be used to guide positioning, orientation and/or activation of examination tools, where the virtual guiding elements may include but are not limited to instruction in the form of plain text or otherwise, one or more arrows, targets, circles, color-changing elements, progress bars, transparent elements, angles, ‘ghost’ outlines of real-world objects, projections of real-time or stored imaging data or overlaid virtual organs, or other virtual items. Furthermore, these guiding elements may also serve to guide position and/or orientation of the user, examinee, or other observer with the intention of helping guide the user through the medical examination in order to achieve improved quality or quantity of data acquired during the examination, reduce inter-operator variability, and/or enable one or more clinicians to do a medical examination that was previously difficult or impossible to perform with good results due to limitations in skill or capability.

At 230 the virtual guiding elements are overlaid as static and/or video elements and are then displayed on a head mounted display or an electronic display device (including but not limited to devices such as tablets, smartphones, and/or laptops). In one embodiment, the guiding elements are simply visible through the display. In another embodiment, these virtual guiding elements may be overlaid on top of a video feed, where the video feed may be that of the live feed of relevant view(s) for performing the medical examination. In one embodiment in which a head-mounted display is utilized, virtual guiding elements may simply be displayed, with visualization of the environment achieved because the head-mounted display may be see-through.

At 232 the system may optionally determine if the user has moved the detector and/or other tools, bodies, or parts thereof to the desired pose(s) and/or location(s). This will be performed using none, one or more of manual input such as that provided by the user, computer vision analysis such as visual odometry and/or visual inertial odometry, analysis of sensor data such as accelerometers and/or gyroscopes, and/or other methods known to one skilled in the art. If the user has failed to place the detector, for example, in the desired location, the system will proceed to step 226 to reassess the current detector pose relative to the optimal and/or desired pose and continue through steps 228 and 230 to regenerate and/or update virtual guiding elements. The user may be allowed some amount of time to achieve the desired performance with the updated guiding elements before the system may reassess the user's performance again at step 232. This may be done until the system determines the detector has been adequately placed, which may be determined in any number of ways via methods known to one skilled in the art. For example, one may utilize a distance measurement from the desired detector pose to the actual detector pose, with an acceptable distance threshold provided in the exam instructions.

At step 234, the system may optionally apply settings adjustments and/or additional data acquisition features including but not limited to adjustments in gain, depth of penetration, frequency, color flow Doppler, pulse wave Doppler, continuous wave Doppler, 3D ultrasound modes, A-mode, B-mode, and/or M-mode. For clarification, here, Doppler refers to a change in perceived frequency when a detector (or observer) moves relative to a wave source. This technique is commonly utilized to gather data about blood flow, as the blood flows through chambers of the heart and is moving relative to a stationary ultrasound probe. In the instance of a cardiac examination, adjusting the frequency might help improve image resolution, for example, while Doppler might help determine if there is adequate blood flow from the left ventricle into the aorta (i.e. ejection fraction) or whether blood is transiting a section of a vessel at a higher-than-expected velocity, which may be used as an indicator of stenosis causing a reduced vessel lumen radius that section of the vessel.

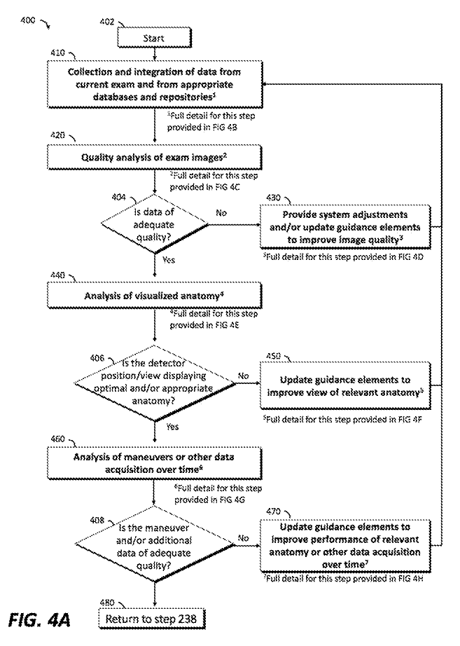

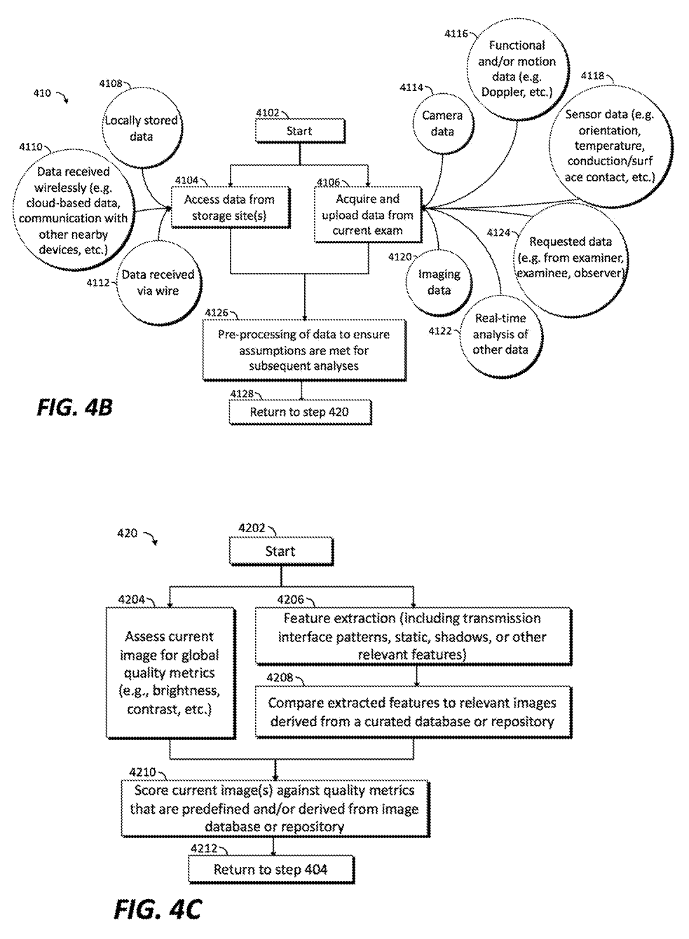

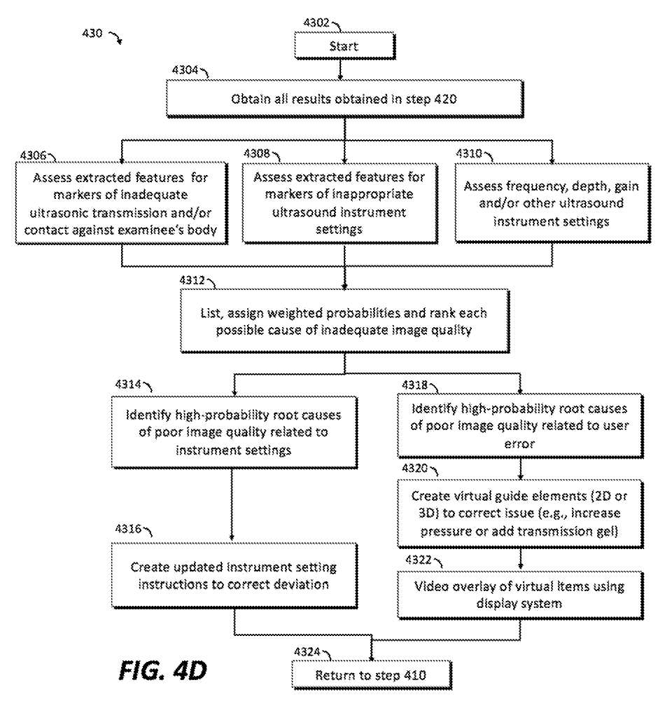

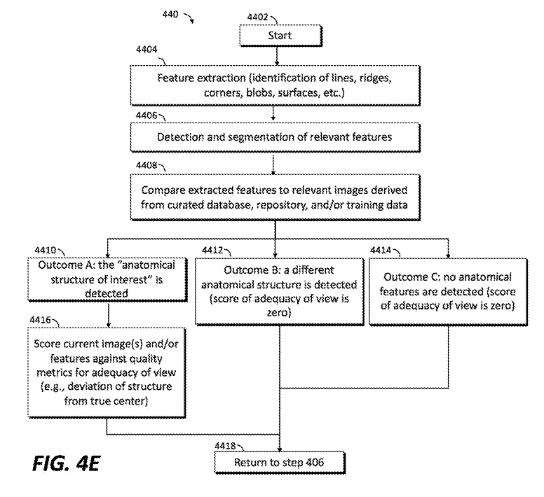

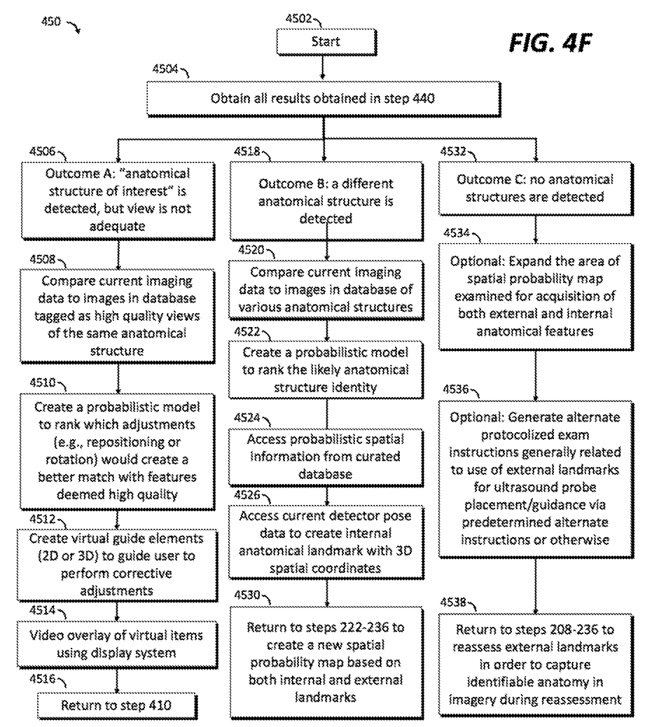

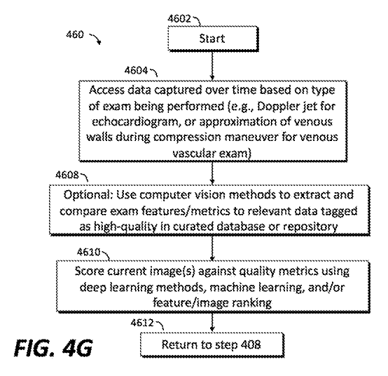

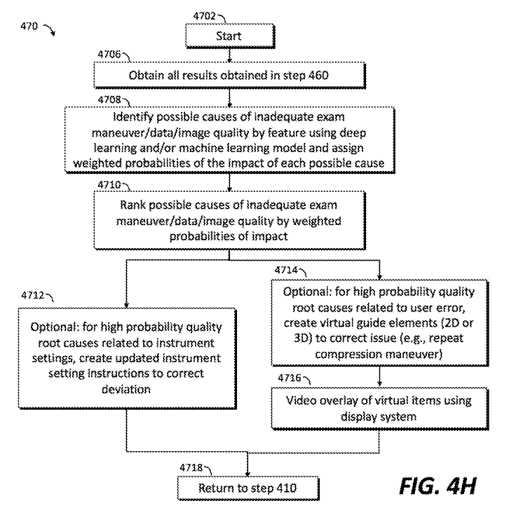

At 236, imaging data and/or other examination data may be captured and uploaded. This imaging data and/or other examination data (e.g. examinee positioning, Doppler, etc) may be either stored locally on an image capture device or sent by the system to the cloud or other storage system either through a wire or wirelessly. The system may then optionally employ step 400, which utilizes real-time analysis of data to evaluate data quality and/or generate additional guidance elements. This data may include but is not limited to images and/or video collected via the detector or otherwise, one or more accelerometers, inertial data, and/or one or more gyroscopes. Examples of real-time exam data and/or performance may include but are not limited to detector data such as images, video, functional data and/or sensor data, user, examinee, or observer positioning, lighting, medical examination data, orientation of one or more involved devices, orientation of user, examinee, or observer, time since examination start, or input from a user, examinee, or observer. In one embodiment, data is collected and utilized purely in a local system. In another embodiment, interpretation of data via a remote entity such as an observer or cloud-based processing unit may be utilized. Additional detail for step 400 is provided in FIG. 4, including methods employed.

At step 238, the system determines if an examination maneuver is required. This may be determined in any number of ways, including but not limited to predefined thresholds or exam instruction such as those accessed in step 206. If the system determines that a maneuver is required, guiding elements are generated to aid performance of the exam maneuver in step 240. In one embodiment, the chosen exam may be a vascular exam and the desired maneuver may be venous compression. This maneuver may include pressing an ultrasound probe down onto the examinee's skin and underlying tissue to see if an underlying vessel compresses. In the case of deep vein thrombosis, the vein would not compress if a clot is present; however, if no clot is present the vein would compress. In such an embodiment, the guiding elements generated may aid in directional compression of the femoral vein. In another embodiment, the chosen exam may be a gallbladder exam, and the desired maneuver may be one aiming to elicit a “sonographic Murphy's sign”. In such an embodiment, the guiding elements generated may aid in directional compression resulting in contact of an inflamed gallbladder with peritoneum in the case of acute cholecystitis. In the event that this maneuver caused examinee discomfort and/or inspiratory arrest, it would indicate a positive “sonographic Murphy's sign.” Step 240 may generate guiding elements for any number of examination maneuvers and it should be understood that the listed embodiments are provided as examples and should in no way be limiting to the scope of possible exam maneuvers encompassed.

At step 242, the system determines if additional views are required to complete the examination. This may be determined in any number of ways. In one embodiment, predefined exam instruction such as those accessed in step 206 are used to determine the need for additional views. In another embodiment, the system determines whether or not additional views are required by measuring examination data for quality and/or through assessment of the exam via various sensors, such as a camera. The assessment and/or measurement of the data may be done via a cloud-based processing unit or local electronic device. In another embodiment, this is done manually by the user and/or observer. And in yet another embodiment, a combination of manual interpretation and electronic device and/or cloud-based unit processing is performed. If additional views are required, the system may optionally employ step 246 to reposition the patient. Patient repositioning may be determined in any number of ways, including but not limited to predefined exam instructions such as those accessed in step 206 and/or a combination of predefined instructions and dynamic decision making based upon other system-generated or collected data such as probability maps generated in step 222 or poor-quality results found in step 400, for example. If there are no additional views required to complete the examination, the exam is ended at step 244.

FIG. 3 is a flow chart depicting detail of step 300 in FIG. 2, which employs computer vision techniques and/or sensor inputs to identify relevant examinee position and/or anatomical landmarks.

Method 300 may be performed locally on an electronic device, online via a cloud system, or via some combination of the two. The method begins at 302. The system may employ none, one, or both of a computer vision based methodology and a sensor and/or device based methodology. The computer vision based methodology begins at step 304, where data is acquired from sensors including but not limited to cameras, range sensors, tomography devices, radar, ultra-sonic cameras, etc. In one embodiment, a simple camera is used. In another embodiment, two cameras are used in order to acquire depth information about a 3D environment. At step 306, pre-processing is performed to ensure acquired data satisfies method assumptions. For example, the system may use incoming data in a calibration step to determine whether or not the incoming sensor data satisfies the system's assumptions pertaining to acquisition of data.

At 308, the system may optionally extract features including, but not limited to lines, ridges, corners, blobs, surfaces, etc, using techniques known to one skilled in the art such as edge detectors, for example. At step 310 the system may optionally utilize these features to create assemblies of features segmented into categories or groups. For example, several detected lines may be grouped together to form a vessel wall or the ventricle of the heart. At step 312 the system may optionally take these grouped and/or categorized features and identify them as relevant landmarks. For example, the system may take a grouping of lines forming of vessel wall and identify them as the femoral vein, or a group of rapidly moving lines and identify them as the outer wall of the left ventricle.

The sensor and/or device based methodology begins at step 314, where the user is prompted to place sensor(s) and/or device(s) at relevant anatomical landmarks. In one embodiment, the system may use an ultrasound detector probe as the device, and the relevant landmark may be the sternal notch. In such an embodiment, step 314 may prompt a user to place the ultrasound detector probe on the examinee's sternal notch. At step 316, the system may then register spatial location and/or input from device(s) and/or sensor(s). In the described embodiment, step 316 may register the spatial location of the ultrasound detector probe placed at the sternal notch. At step 318, the system may then detect zero, one or more of gestures, voice commands, eye movements, device and/or sensor input, and/or other user interface input to confirm and/or modify spatial registration of landmarks. In the described embodiment, the user may press a button on the ultrasound detector probe when it is properly placed on the sternal notch. In another embodiment, the user may speak a phrase, such as “confirm landmark” or “finalize.” In another embodiment, the user may make a fist with digits two, three, four, and five, with digit five located most inferior, followed by digits four, three, and two, with his or her thumb/first digit located superiorly and pointed in an upward motion.

At step 320, the system may optionally cross-compare landmark identification data culminating from steps 304-312 with pose data and/or spatial data from steps 314-318 in the event that both computer vision and sensor and/or device based methodologies are utilized. This step allows the system to rectify differences in the event that it acquires conflicting data. At step 322 the system may optionally resolve any inconsistencies found in step 320. At step 324, the system may optionally display a virtual landmark map to the user, identifying important landmarks and where the system believes each is located. At step 326, the system may optionally prompt the user for input to confirm or modify the virtual landmark map presented in step 324. At step 328, the system may then consolidate the information gathered in prior steps to finalize the landmark map data. For example, the user may make adjustments to the location of the examinee's xyphoid process in step 326, and in step 328 the system would use this adjustment to make modifications to its working model of landmark map data. At step 330, the system returns to step 216 outlined in FIG. 2.

FIG. 4A is a flow diagram showing detail of step 400 from FIG. 2, in which data collected from a detector is analyzed in real time and the analysis is used to update virtual exam guidance elements.

Method 400 may be performed locally on an electronic device, online via a cloud system, or via some combination of the two. The method begins at 402. At step 410, a range of data inputs are collected and integrated from the current exam (for example, the real-time images collected by an ultrasound detector) and from appropriate databases and repositories. Further detail on step 410 is provided in FIG. 4B. At step 420, the quality of the images being collected by the exam detector (for example, an ultrasound probe) are evaluated to determine whether the technical quality of the images is sufficient to allow further assessment and interpretation. Further detail on step 420 is provided in FIG. 4B. At step 404, the system accesses the results of the analysis performed in step 420 to determine if the image quality has met a pre-determined threshold. This threshold may be determined in any number of ways known to one skilled in the art. For example, in one embodiment, the threshold may be based on images stored in a database that have been rated by individuals skilled at ultrasound interpretation. If the threshold for adequate image quality has not been met, then the system enacts adjustments and/or updates virtual exam guidance elements in step 430, as described in detail in FIG. 4D, after which the system returns to step 410. If the threshold for adequate image quality has been met in step 404, then the system proceeds to step 440 where the system analyzes anatomical structures that appear in the images collected by the detector (for example, an ultrasound probe), as described in detail in FIG. 4E. At step 406, the system accesses the results of the analysis performed in step 440 to determine if the appropriate anatomical structures are present and/or if the detector position/view is optimal. The identity of anatomical structures and the positioning of an optimal view may be determined in any number of ways. For example, in one embodiment this information may be based on images stored in a database that have been rated by individuals skilled at ultrasound interpretation. If the required anatomical structures are not present in the field of view, or if the view of the target anatomical structures is not optimal, then the system may update virtual exam guidance elements in step 450, as described in detail in FIG. 4D, after which the system returns to step 410. If the required anatomical structures are present in the field of view, and if the view of the target anatomical structures is optimal, then the system proceeds to step 460 where the system analyzes data that are captured over time (for example, Doppler flow detection or the response of anatomical structures during a maneuver such as compression), as described in detail in FIG. 4G. At step 408, the system accesses the results of the analysis performed in step 460 to determine if the data collected during a maneuver and/or other data collected over time have met a pre-determined threshold. This threshold may be determined in any number of ways known to one skilled in the art. For example, in one embodiment this threshold may be based on images stored in a database that have been rated by individuals skilled at ultrasound interpretation. If the threshold for adequate data quality has not been met, then the system may update virtual exam guidance elements in step 470, as described in detail in FIG. 4H, after which the system returns to step 410. If the threshold for adequate data quality has been met, then the system reaches step 480, in which the method described in FIG. 2 is resumed at step 238.

FIG. 4B is a flow diagram showing detail of step 410 from FIG. 4A, in which necessary data inputs are collected and integrated. Method 410 may be performed locally on an electronic device, online via a cloud system, or via a combination of the two. The method begins at 4102. At step 4104, data is accessed from appropriate storage sites, including locally stored data 4108, data received wirelessly (for example, cloud-based data) 4110, and/or data received by wire 4112, including but not limited to images from prior exams that have been rated for image quality, identity of anatomical structures, quality of view (for example whether the structure is in the middle of the detector's field of view), or adequacy of maneuver performance (for example, if a compression maneuver fully approximates the walls of a vein). At step 4106, the system acquires and uploads data from the current exam, including data from the camera 4114 mounted on a tablet or head-mounted display device, functional and/or motion data 4116 (for example, ultrasound images collected with Doppler flow analysis), sensor data 4118 (for example, gyroscope or accelerometer data), requested data provided by manual input 4124 (for example, inputs from the examiner/user, examinee or third-party observer), real-time analysis of other data 4122 or imaging data 4120 (for example, ultrasound images collected in real time). At step 4126, these data are pre-processed to ensure that basic assumptions are met for subsequent analysis. For example, the system may use incoming data in a calibration step to determine whether or not the incoming sensor data satisfies the system's assumptions pertaining to acquisition of data. At step 4128, the system returns to step 420 described in FIG. 4A.

FIG. 4C is a flow diagram showing detail of step 420 from FIG. 4A, in which exam image quality is assessed. Method 420 may be performed locally on an electronic device, online via a cloud system, or via a combination of the two. The method begins at 4202. At step 4204, the system assesses the current image for global quality metrics (e.g., brightness, contrast etc.). For example, the system may assess contrast using absolute mean brightness error, entropy, and/or another relevant metric. At step 4206, the system analyzes the current ultrasound image and extracts key features related to image quality, such as shadowing signatures, static signatures, echogenic signatures that represent the contact points between the ultrasound probe, transmission gel, examinees skin and/or other relevant features. At step 4208, the system compares the extracted features from step 4206 to features that may be derived from comparison images stored in a database or repository and/or that have been assessed by individuals skilled at ultrasound interpretation. At step 4210, the system may integrate global image assessment metrics from 4204 and feature comparisons against stored images from 4208 to create an integrated score of the current image quality using a pre-defined threshold and/or threshold(s) derived from cumulative assessment across images in a database or repository. Alternately, the system may employ machine learning methods known to one skilled in the art. For example, a machine-learning algorithm could be designed to function as an integrated classifier and grade images as “adequate” or “inadequate” based on features learned from a training database composed of images previously assessed by individuals skilled in ultrasound interpretation, which would consolidate steps 4204-4210 into a single step. At step 4212, the system returns to step 404 described in FIG. 4A.

FIG. 4D is a flow diagram showing detail of step 430 from FIG. 4A, in which the method provides system adjustments and/or updated guidance elements to improve image quality. Method 430 may be performed locally on an electronic device, online via a cloud system, or via a combination of the two. The method begins at 4302. At step 4304, the system obtains all results from step 420, including the results of feature extraction in step 4206. At step 4306, the system assesses extracted features for signatures and/or markers of inadequate ultrasonic transmission and/or contact against the examinee's body. This may be done in any number of ways known to one skilled in the art. For example, the system may perform this step by comparing features of the current images to stored images that have been interpreted by individuals skilled at ultrasound interpretation. Examples of features may include a signature that correlates with the interpretation of inadequate amount of transmission gel, inadequate contact between the ultrasound probe and examinees skin and/or other scenarios that could degrade the quality of the image. At step 4308, the system assesses extracted features for markers of inappropriate ultrasound instrument settings by comparing features of the current images to stored images such as those that have been interpreted and/or sorted by individuals skilled at ultrasound interpretation. For example, the system may detect a signature that correlates with inappropriate settings for the frequency of ultrasonic transmission, the depth of the recorded image, gain settings and/or other ultrasound instrument settings that could affect the quality of the image. At step 4310, the system creates a record of current ultrasound instrument settings (for example frequency, depth and gain). At step 4312, the data collected in steps 4306, 4308, 4310, are integrated into a list of possible causes of inadequate image quality, which the system then ranks according to weighted probabilities of the likelihood that each possible cause could be contributing to inadequate image quality. Alternately, machine learning may be employed using techniques known to one skilled in the art. For example, an algorithm could be designed to function as an integrated classifier to identify and rank possible root causes based on features learned from a training database composed of images previously assessed by individuals skilled in ultrasound interpretation, which would consolidate steps 4304-4312 into a single step. At step 4314, the system identifies high-probability root causes of poor image quality related to instrument settings, and at step 4316, the system creates updated instrument setting instructions to correct the deviation. In some cases, these instructions may be enacted automatically by the system, while in other cases, these instructions may be communicated to the user via 2D or 3D virtual guidance elements. At step 4318, the system identifies high-probability root causes of poor image quality related to user error, and at step 4320, the system creates virtual exam guidance elements (2D or 3D) to correct one or more of these root causes. For example, if the system identified inadequate amounts of transmission gel as a high-probability cause of poor image quality, the virtual exam guidance element would communicate to the user that additional transmission gel should be added. At step 4322 the virtual guidance elements generated in step 4320 are provided in a video overlay using a display system to present the guidance elements to the user. At step 4324, the system returns to step 410 described in FIG. 4A.

FIG. 4E is a flow diagram showing detail of step 440 from FIG. 4A, in which visualized anatomy is analyzed. Method 440 may be performed locally on an electronic device, online via a cloud system, or via a combination of the two. The method begins at 4402. At step 4404, the system analyzes the current ultrasound image and extracts key features related to visualized structures, including but not limited to lines, ridges, corners, blobs, surfaces, echogenic interfaces, etc. using techniques known to one skilled in the art such as edge detectors, for example. At step 4406 the system may utilize these features to create assemblies of features segmented into categories or groups. For example, several detected lines may be grouped together to form a vessel wall or the ventricle of the heart. At step 4408 the system takes these grouped and/or categorized features and identifies them as relevant anatomical landmarks. This may be done in any number of ways known to one skilled in the art. In one embodiment, the system may take these grouped and/or categorized features and compare them to features derived from images stored in a database or repository that have been assessed by individuals skilled at ultrasound interpretation. For example, the system may take a grouping of lines forming a vessel wall and identify them as the femoral vein, or a group of rapidly moving lines and identify them as the outer wall of the left ventricle. Based on this assessment, 3 main outcomes are possible. At step 4410, the system determines that the result of the comparison is outcome A: that the “anatomical structure of interest” has been detected. The identity of the “anatomical structure of interest” may be determined by the exam instructions identified in FIG. 2, step 206, and may then be cross-compared with the identities of anatomical structures such as those defined in a repository that has been assessed by individuals skilled in ultrasound interpretation. Under this condition, the system proceeds to step 4416, where the current image is scored for quality metrics related to the adequacy of the view. For example, the structure of interest may be located at the edge of the current view of the ultrasound probe, and would be scored as having an inadequate view based on the degree of deviation from the true center of the probe's field of view. At step 4412, the system determines that the result of the comparison is outcome B: that an anatomical structure other than the “anatomical structure of interest” has been detected, and as a result the score of the adequacy of the current view is zero. At step 4414, the system determines that the result of the comparison is outcome C: that no identifiable anatomical structures can be identified, and as a result the score of the adequacy of the current view is zero. Alternately, the system may employ machine learning methods known to one skilled in the art. For example, the algorithm could be designed to function as an integrated classifier to identify and rank the probability of outcomes A, B and C based on features learned from a training database composed of images previously assessed by individuals skilled in ultrasound interpretation, which would consolidate steps 4410-4416 into a single step. After the system determines whether outcome A, B or C is most probable, it progresses to step 4418, which involves returning to step 406 in FIG. 4A.

FIG. 4F is a flow diagram showing detail of step 450 from FIG. 4A, in which the method provides updated guidance elements to improve the view of relevant anatomy. Method 450 may be performed either locally on an electronic device, online via a cloud system, or via a combination of the two. The method begins at 4502. At step 4304, the system obtains all results from step 440, including the results of feature extraction, feature detection and segmentation, and comparison to images derived from any curated databases in steps 4410-4414, as well as the determination of whether the current view is best described by outcome A: the “anatomical structure of interest” is detected, outcome B: a different anatomical structure is detected or outcome C: no identifiable anatomical structure is detected.

At step 4506, the system has used method 440 to determine that outcome A has occurred (i.e., that the “anatomical structure of interest” is detected), but that the view was not adequate. At step 4508, the system may compare the current image to images in a database, such as those previously identified by individuals skilled at ultrasound interpretation as high-quality views of the same anatomical structure. For example, the popliteal vein might be the “structure of interest” for one portion of a lower extremity venous exam, and after the first placement of the ultrasound probe it may appear at the edge of the ultrasound probe's field of view whereas the images in the database identified as high-quality views show the popliteal vein in the center of the probe's field of view. At step 4510, the system creates a probabilistic model to rank which adjustments would create a better match with features that are deemed high quality. Such high-quality features may be derived from images/views in a database that were previously identified as high-quality. In the previously referenced example, the popliteal vein is at the edge of the ultrasound probe's field of view, and the system may determine that a lateral repositioning of the probe would move the popliteal vein into the middle of the ultrasound probe's field of view. In one embodiment, the system may determine this by noting such a repositioning would result in a view consistent with a comparator image or images drawn from a database previously identified as containing high-quality views. As an alternate example, the adjustment with the highest probability of generating a high-quality view might be a rotation movement. At step 4512, the system translates the preferred adjustment into 2D or 3D virtual exam guidance elements designed to guide the user to perform the preferred adjustment. For example, an augmented reality arrow might be displayed in 3D space labeled with the distance (e.g., “1.3 cm”) of the desired repositioning movement. In this example, the system might also generate an augmented reality ‘ghost’ outline of a probe showing the desired position of the probe in 3D space. As an alternate example, the system could generate a rotational arrow labeled with the number of degrees (e.g., “76°”) to guide the user to adjust the probes position by rotating it. At step 4514, one or more 2D or 3D virtual exam guidance elements are displayed using the display system, which may be an electronic display device such as a tablet, see-through head-mounted display, projection system and/or other display system. At step 4516, the system returns to step 410 described in FIG. 4A.

At step 4518, the system has used method 440 to determine that outcome B has occurred, (i.e., that an anatomical structure other than the “anatomical structure of interest” is detected. At step 4520, the system may compare the current image to images of various anatomical structures in a database previously identified by individuals skilled in ultrasound interpretation. At step 4522, the system creates a probabilistic model to rank the likely identity of the anatomical structure that is present in the image from the current exam. For example, the “anatomical structure of interest” could be the abdominal aorta for an abdominal aortic aneurysm screening exam, but the initial placement of the probe could instead visualize a different anatomical structure that has a high probability of being the examinee's kidney, which, in one embodiment, by be determined by comparison to a database containing images of a range of anatomical structures including aortas and kidneys. At step 4524, the system accesses probabilistic relative spatial data from the curated database. In the previously referenced example, the database would contain spatial data relating to the likely position of a kidney relative to other internal and/or external landmarks, which, in one embodiment, may be based on multiple previously-imaged kidneys. At step 4526, the system accesses current detector pose data. At step 4528, the system uses data from steps 4524 and 4526 to create an internal anatomical landmark with 3D spatial coordinates. At step 4530, the system returns to step 222 as described in FIG. 2, and may incorporate the newly-created internal anatomical landmark into the probability map for potential detector poses that is utilized throughout steps 222-236.

At step 4532, the system has used method 440 to determine that outcome C has occurred, (i.e., that no identifiable anatomical structure is detected. At step 4534, the system optionally expands the area of the spatial probability map that was initially constructed in step 222 in FIG. 2, such that a wider variety of optimal probe poses is considered. At step 4536, the system optionally generates an alternate set of protocolized exam instructions for ultrasound probe placement/guidance. For example, the initial protocolized exam instructions for an abdominal aortic aneurysm screening exam may have included placement of the probe in one location between the xyphoid process and the navel; however, if no identifiable anatomical structures are visualized after the initial placement of the ultrasound probe, the alternate protocolized exam instructions may include a “sweep” maneuver to collect data at multiple positions between the xyphoid and the navel such that at least one collected image may contain an identifiable anatomical structure. At step 4538, the system returns to steps 208-236 described in FIG. 2 in order to capture identifiable anatomy through the performance of a modified assessment.

FIG. 4G is a flow diagram showing detail of step 460 from FIG. 4A, in which the method analyzes maneuvers and/or other data acquisition over time. Method 460 may be performed locally on an electronic device, online via a cloud system, or via some combination of the two. The method begins at 4602. At 4604, the system accesses data captured over time based on the type of examination being performed. For example, the system may capture Doppler jet flow during an echocardiogram, or video displaying approximation of the venous walls relative to one another during a compression maneuver performed during a vascular examination. At step 4606, the system may optionally utilize deep learning and/or computer vision methods known to one skilled in the art to extract and/or compare exam features and/or metrics to relevant data tagged as “high-quality” in a curated database or repository. For example, the system may collect Doppler data of a vessel indicating that the ultrasound detector probe is skewed, rather than taking a perpendicular cross-section of a vessel. In step 4606, the system may compare this Doppler data with Doppler data from “high-quality” data from a curated database and/or repository and note the discrepancy. In another embodiment, the system may utilize deep learning methods to allow the system to classify its own features associated with “high-quality” data. At step 4608 the system may score the acquired image(s)/data against quality metrics using feature/image ranking or through the use of machine learning methods, such as a model trained on a curated database of high-quality images and/or associated features, for example. At step 4610 the system returns to step 408 as outlined in FIG. 4A.

FIG. 4H is a flow diagram showing detail of step 470 from FIG. 4A, in which the method updates guidance elements to improve performance of relevant anatomy or other data acquisition over time. Method 470 may be performed locally on an electronic device, online via a cloud system, or via some combination of the two. The method begins at 4702. At step 4704, the system obtains all the results obtained in step 460 as detailed in FIG. 4G. At step 4706, the system identifies possible causes of inadequate exam maneuver(s)/data/image quality by feature and assigns weighted probabilities of the impact of each possible cause. In one embodiment, the system may utilize deep learning, allowing the system to extract its own set of features pertaining to exam maneuver(s)/data/image quality. In another embodiment, the system may utilize machine learning techniques known to one skilled in the art. For example, the system may utilize a machine learning model trained using a repository of “high-quality” data with features tagged that may serve as good indicators of quality. In another such embodiment, features may be extracted without the use of machine learning and assigned weighted probabilities based on the impact each feature may have on quality. Such probabilities may be assigned and/or derived any number of ways, such as through machine learning methods, via comparison with repository data, and/or through predetermined probability weights, for example.

At 4708, the system may optionally rank possible causes of inadequate exam maneuver(s)/data/image quality by weighted probabilities if assigned in step 4706. At step 4710, the system may optionally identify quality root causes related to instrument and/or device settings, such as those that may have been given high probability rankings in step 4708, and create updated instrument setting instructions to correct quality and/or deviations. These instructions may be created through various methods known to one skilled in the art such as deep learning methods, machine learning methods, and/or via predetermined thresholds and/or instructions. The instructions created in step 4710 may be used to automatically adjust instrument/device settings and/or presented to the user for manual adjustment.

At step 4712, the system may optionally identify quality root causes related to user error, such as those that may have been given high probability rankings in step 4708, and create virtual guide elements (2D or 3D) to correct the issue. For example, the system may identify that a compression maneuver was performed poorly and prompt the user to repeat the maneuver or make a small adjustment to his or her technique using other guide elements. At step 4714, the system provides a video overlay of the virtual items generated in step 4708 using the display system in order to convey the virtual guide elements and/or prompting to the user. At step 4716, the system returns to step 410 as detailed in FIG. 4A.

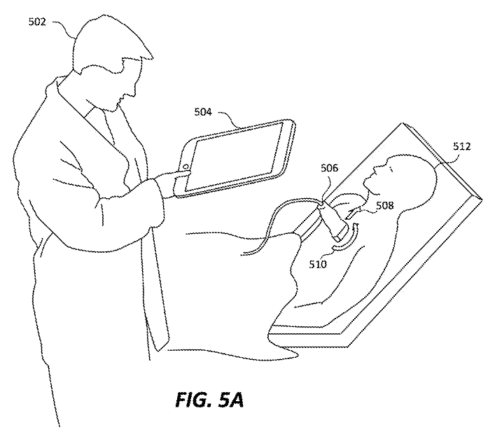

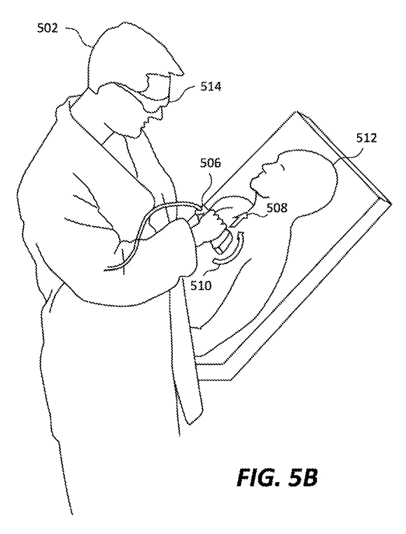

FIGS. 5A and 5B are illustrations depicting two embodiments of a method for guiding a medical examination, wherein the medical examination is a cardiac ultrasound exam (echocardiogram).

FIG. 5A is an illustration of an embodiment of the system and method in which virtual guidance is provided via an electronic display device for the performance of a cardiac ultrasound exam (echocardiogram). In this illustration, the user 502 is holding an electronic display device 504 that is capable of displaying the virtual guiding elements 508 and 510 in augmented reality. The electronic display device 504 may use a camera to display the imagery behind the device and simultaneously augment the imagery with virtual guiding elements. Thus, the user would simply view the world through the electronic display device 504 while performing the exam, and the guiding elements displayed via the electronic display device would aid the user in performing the exam.

FIG. 5B is an illustration of an embodiment of the system and method in which virtual guidance is provided via a see-through head mounted display device for the performance of a cardiac ultrasound exam (echocardiogram). While a see-through head mounted display 514 is depicted, the same principle applies to other head mounted displays that may not be see-through.

In both FIG. 5A and FIG. 5B, the user 502 holds an ultrasound probe or transducer 506 near the relevant anatomy of the examinee 512. The relevant anatomy of the examinee 512 in this instance might include the space between the 5^{th} and 6^{th} ribs, which confers an adequate window of the heart from the apex. In this illustration, the user 502 has the ultrasound probe 506 positioned caudal and inferior to the desired anatomic location. As described in detail in FIGS. 2, 3, and 4A-H, in order to generate virtual guiding elements, the system accesses any of a number of data sources, including downloaded exam instructions, spatial identification of examinee anatomical landmarks, pose data from the camera, pose data from the ultrasound probe or other sensor systems, and/or real-time image analysis. These data inputs are a non-exhaustive list; for a detailed description of data inputs and the full methodology by which virtual exam guiding elements are generated, refer to FIGS. 2, 3, and 4A-H. In this illustration, the system has generated virtual exam guidance elements including a directional arrow 508 and a rotational arrow 510 to direct the examiner to reposition the ultrasound probe 506.

Similarly, a user may use examination tools other than an ultrasound probe along with guiding elements to perform other kinds of exams. Furthermore, virtual guiding elements other than those depicted herein may be utilized. The guiding elements illustrated are given to demonstrate the general concept of guidance. The ultrasound probe 506 may be wired or wireless. In FIGS. 5A and 5B, the electronics display device 504 and head mounted display 514 may each display guiding elements, examination tool imaging, and/or video, as well as perform some or all of the processing as outlined in FIGS. 2, 3, and 4A-H.

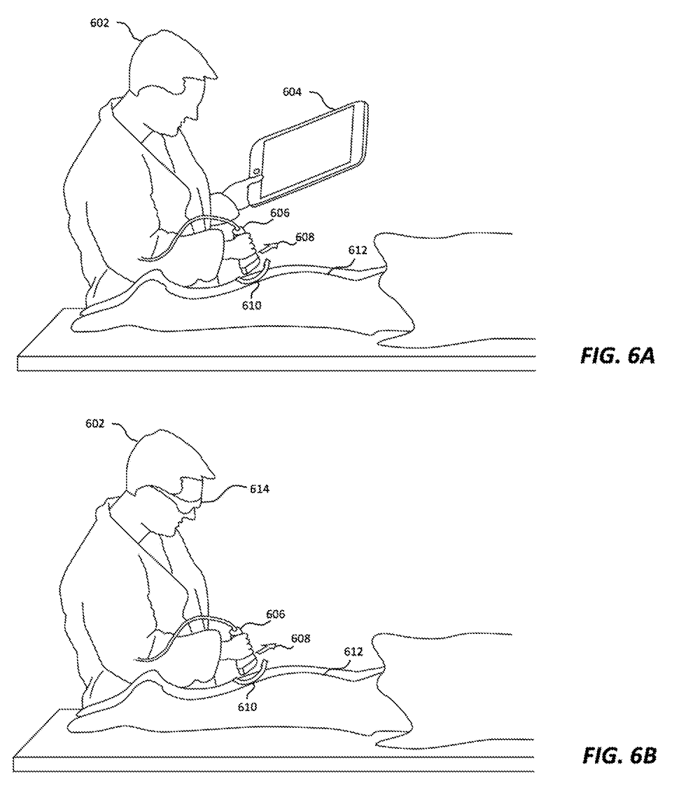

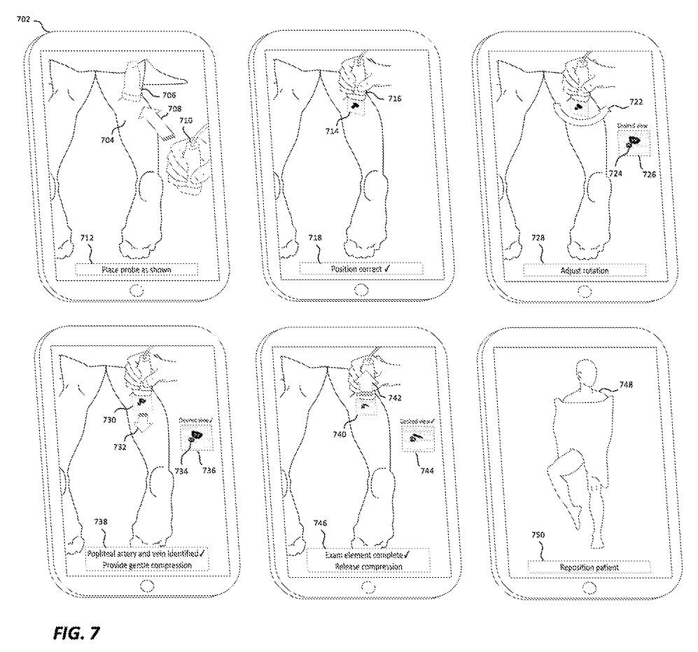

FIGS. 6A and 6B are illustrations depicting two embodiments of a method for guiding a medical examination, wherein the medical examination is a vascular examination.

FIG. 6A is an illustration of an embodiment of the system and method in which virtual guidance is provided via an electronic display device for the performance of a cardiac ultrasound exam (echocardiogram). In this illustration, the user 602 is holding an electronic display device 604 that is capable of displaying the virtual guiding elements 608 and 610 in augmented reality. The electronic display device 604 may use a camera to display the imagery behind the device and simultaneously augment the imagery with virtual guiding elements. Thus, the user would simply view the world through the electronic display device 604 while performing the exam, and the guiding elements displayed via the electronic display device would aid the user in performing the exam.

FIG. 6B is an illustration of an embodiment of the system and method in which virtual guidance is provided via a see-through head mounted display device for the performance of a vascular exam. While a see-through head mounted display 614 is depicted, the same principle applies to other head mounted displays that may not be see-through.

In both FIG. 6A and FIG. 6B, the user 602 holds an ultrasound probe or transducer 606 near the relevant anatomy of the examinee 612. The relevant anatomy of the examinee 612 in this instance might include the popliteal vein, which runs behind the knee. In this illustration, the user 602 has the ultrasound probe 606 positioned caudal and inferior to the desired anatomic location. As described in detail in FIGS. 2, 3, and 4A-H, in order to generate virtual guiding elements, the system accesses any of a number of data sources, including downloaded exam instructions, spatial identification of examinee anatomical landmarks, pose data from the camera, pose data from the ultrasound probe or other sensor systems, and/or real-time image analysis. These data inputs are a non-exhaustive list; for a detailed description of data inputs and the full methodology by which virtual exam guiding elements are generated, refer to FIGS. 2, 3, and 4A-H. In this illustration, the system has generated virtual exam guidance elements including a directional arrow 608 and a rotational arrow 610 to direct the examiner to reposition the ultrasound probe 606.

Similarly, a user may use examination tools other than an ultrasound probe along with guiding elements to perform other kinds of exams. Furthermore, virtual guiding elements other than those depicted herein may be utilized. The guiding elements illustrated are given to demonstrate the general concept of guidance. The ultrasound probe 606 may be wired or wireless. In FIGS. 6A and 6B, the electronics display device 604 and head mounted display 614 may each display guiding elements, examination tool imaging, and/or video, as well as perform some or all of the processing as outlined in FIGS. 2, 3, and 4A-H.

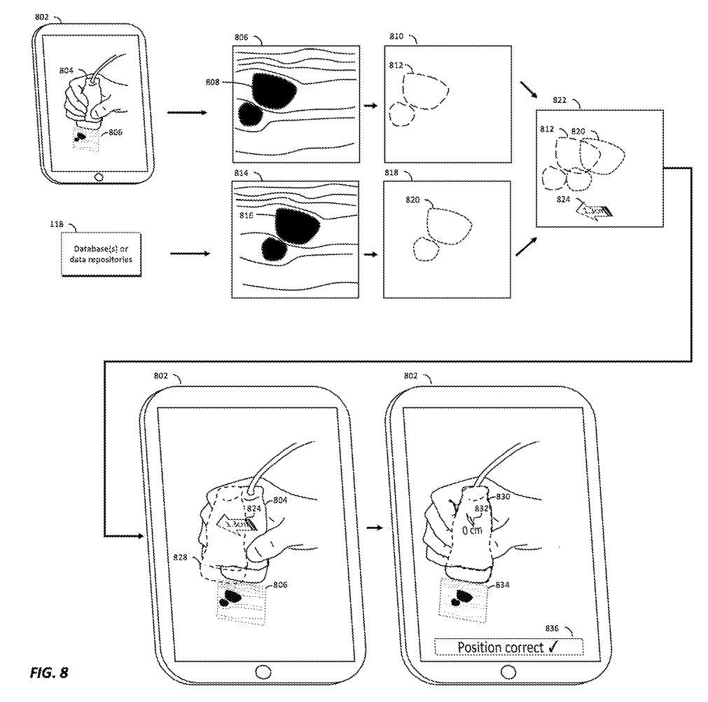

FIG. 7 is an illustration of an embodiment of the digital or virtual guidance elements described in the disclosed method, where the guidance elements are designed to aid the user in performing a lower extremity vascular exam. FIG. 7 further illustrates the view a user might see as outlined in FIG. 6A through an electronic display device 702, along with several possible embodiments of virtual guidance instructions a user may receive while performing a vascular examination. For example, the user may be instructed to change the position of an imaging detector or other exam instrument via text 712. In the first sequence, the examinee 704 can be seen lying face down, with the ultrasound probe 710 being held in close proximity to the patient. A guiding element in the form of an arrow 708 can be seen directing the user to move the ultrasound probe 710 towards a 3D virtual outline of an ultrasound probe 706 positioned at the desired ultrasound probe location. Such a 3D virtual object demonstrating the desired position of the ultrasound probe may variously be called a ‘ghost’ outline of where the real physical probe should be placed. While this sequence and relevant virtual exam guidance elements are specific to a vascular ultrasound examination, the principles of a ‘ghost’ outline of a virtual examination tool and a directional arrow may apply to many different medical examinations.

The following sequence demonstrates the use of text instruction and virtual imagery. The user is told via text “position correct” 718, with the ultrasound probe 716 in the correct position. With the ultrasound probe 716 in position, virtual ultrasound imaging 714 is then displayed, showing the relevant anatomy. The third sequence demonstrates the use of rotational guidance and desired view to aid in medical examination. The user is told via text to “adjust rotation” 728, and a guiding element in the form of a rotational arrow 722 is displayed indicating the direction the ultrasound probe should be turned. A picture of the desired view 726 and relevant anatomy 724 is shown on the screen for reference so that the user may attempt to match it. While a desired view might guide imaging examinations, other metrics might include desired decibels for examinations utilizing audio, or desired variability for examinations, such as blood pressure measurements, where reducing variation is important.

Once the user correctly performs the instructions, he or she is notified via text “popliteal artery and vein identified, provide gentle compression,” 738, indicating that the user successfully followed the prior instruction and providing a new one. This sequence demonstrates the use of maneuver instructions in aiding examination. Again, the ultrasound imaging is displayed 730, along with a new guiding element 732 indicating that the user should compress the vein where the ultrasound is located. The desired view 736 is maintained from the prior sequence along with relevant anatomy 734 to help the user maintain the correct position and rotation of the ultrasound probe.

The user is then shown the new desired view 744 in the following sequence, which has been updated to reflect the desired compression maneuver. Text instructions 746 indicate that the exam element has been completed, and instruct the user to “release compression.” The ultrasound imaging 740 continues to be displayed, and a new guiding element 742 appears, indicating to the user that they may release the applied compression. The guiding elements 732 and 742 demonstrate ways in which an exam maneuver may be guided; however, other possible guidance elements for maneuvers may include, but are not limited to, incorporation of 2D video demonstration or virtual guiding elements such as virtual hands, organs, or other body parts.

The final sequence demonstrates the use of text instruction to reposition the examinee. The user is instructed to “reposition patient” 750. The examinee 748 is seen now lying supine in a position better suited for assessment of vasculature in the inguinal region, such as the femoral artery and vein. It should be noted that although this embodiment was specific to vasculature exams, these principles apply to a wide variety of medical exams that utilize ultrasound, as well as many other medical exams that utilize other examination tools. In combination, the virtual exam guidance elements included in FIG. 7 include arrows, virtual 3D or 2D ‘ghost’ outlines of the desired position of a detector, text instructions, real-time video data displayed in 3D space, exemplary detector data showing the desired view and/or anatomy, anatomical identification and a virtual depiction of patient positioning. These types and/or uses of exam guidance elements are provided as an illustration, and should not be understood to represent the only method of guiding a venous vascular exam, nor should they be understood to represent a comprehensive list of guidance elements that may be deployed to achieve the goal of guiding a high-quality exam.

FIG. 8 is an illustration of how the algorithm described in FIG. 4A-H analyzes current detector-generated images, extracts key features, and creates virtual exam guidance elements. In one embodiment of the invention, also illustrated in FIG. 7, a tablet device 802 is used as the display device, where the user simultaneously views real physical objects, such as the ultrasound probe 804, and virtual objects, such as a real-time 3D augmented reality display 806 of the images that the detector is collecting. In this example, the primary structure of interest is the popliteal vein 808, however in image 806, which the detector is currently collecting, the popliteal vein is not in the center of the field of view. A comparison image 814 is accessed, which may be done utilizing an appropriate database or repository 118, which shows optimal placement of the popliteal vein 816 in the middle of the field of view. According to the algorithm described in FIG. 4E, a feature map is generated for both the current image 810 and the stored comparison image 818. In this example, the identity and position of the popliteal vein is determined by using differences in echogenicity of blood and tissue to identify the circumferential edge of the popliteal vein on both the current real-time image 812 and the comparison image 820. As shown in illustrated frame 822, the system then compares the position of the popliteal vein edges feature in the current view 812 to the desired view in the comparison image 820. The result of this analysis is that the proper structure has been identified, but is not in an optimal position. As described in FIG. 4F, the system then creates a probabilistic model to determine which adjustment would re-align the current view of the popliteal vein 812 to best match the desired view in the comparison image 820, and in this example, the highest rated adjustment is a lateral movement of the probe over a distance of 1.3 cm. The system then uses this data to create virtual exam guide elements that are displayed on the tablet display device 802 to show the user how to adjust the probe. In this example, the virtual exam guidance elements include an augmented reality arrow labeled with the desired distance of the adjustment 824 and an augmented reality ‘ghost’ outline of a probe 828 showing the desired position of the probe in 3D space. The user responds to these instructions by moving the probe in the desired distance, at which point the exam guidance elements are updated to reflect this change, with the arrow 832 shortened and labeled with “0 cm” to show the user that the proper distance has been covered. Once the proper adjustment has been made, the physical probe now completely overlaps with the augmented reality ‘ghost’ probe 830, and the augmented reality projection of the images the ultrasound probe is collecting 834 now shows that the desired view has been obtained. The system also generates a new virtual exam guidance element in the form of a banner with the text “position correct” 836 that communicates to the user that the desired probe position has been achieved.

It should be noted that although this embodiment was specific to vasculature exams, these principles apply to a wide variety of medical exams that utilize ultrasound, as well as many other medical exams that utilize other examination tools. In combination, the virtual exam guidance elements included in FIG. 8 include arrows, virtual 3D ‘ghost’ outlines of the desired position of a detector, text instructions and real-time video data displayed in 3D space. These types and/or uses of exam guidance elements are provided as an illustration, and should not be understood to represent the only method of guiding a venous vascular exam, nor should they be understood to represent a comprehensive list of guidance elements that may be deployed to achieve the goal of guiding a high-quality exam. Furthermore, the analysis depicted in frames 806, 810, 814, 818 and 822 are intended for illustrative purposes only and should not be taken to indicate a comprehensive set of steps required to perform such an analysis, which are described in additional detail in FIGS. 4A-H. It should also be noted that the analysis performed by the method described in FIGS. 4A-H may be performed without creating or displaying images such as those depicted in frames 806, 810, 814, 818 and 822.

The terminology utilized herein is intended to describe specific embodiments of the invention only and is in no way intended to be limiting of the invention. The term “and/or”, as used herein, includes any and all combinations of one or more of the associated listed items. As used herein, the singular forms “a,” “an,” and “the” are intended to include the plural forms as well as the singular forms unless the context clearly indicates otherwise. As used herein, the terms “comprises” and/or “comprising” are intended to specify the presence of features, elements, or steps, but do not preclude the presence or addition of one or more additional features, elements, and/or steps thereof. The term “pose” as used herein should be interpreted as a broad term defined by its plain and ordinary meeting and may include without limitation, position, orientation, or any other appropriate location information. The term “device” as used herein should be interpreted as a broad term defined as a thing adapted for a particular purpose. This includes but is not limited to medical examination tools such as EKG leads, a temperature probe, and/or an ultrasound probe, but also other things a user may utilize in performing an exam such as a user's own hands, ears, nose, or eyes. The term “ultrasound probe” should be understood to mean a hand-held ultrasound transducer and/or detector and/or combination of transducer and detector.

Unless defined otherwise, all terms used herein have the same meaning as commonly understood by one having ordinary skill in the art to which the invention relates. Furthermore, it will be understood that terms should be interpreted as having a meaning that is consistent with their meaning in the context of the relevant art and the present disclosure and will not be interpreted in an overly formal or idealized sense unless expressly defined herein. In order to avoid confusion and lack of clarity, this description will refrain from repeating every possible combination of features, elements, and/or steps. However, it will be understood that any and all of the listed features, elements and/or steps each has individual benefit and can also be used in conjunction with one or more, or in some cases all, of the other disclosed items. Thus, the specification and claims should be read with the understanding that any of these combinations are entirely within the scope of the invention and the claims.

What is claimed is:

1. A health exam guidance system comprising:
a display device configured to present virtual content to a user;
one or more examination devices or sensors configured to collect data related to a medical exam;
one or more processors;
one or more computer storage media storing computer readable instructions which, when executed by the one or more processors, cause the one or more processors to perform operations comprising:
determining a type of medical examination desired;
accessing a set of exam instructions for the determined type of medical exam;
generating one or more virtual elements related to a desired maneuver indicated by the accessed set of exam instructions; and
instructing the display device to present the one or more virtual elements to the user in an augmented reality environment or in a mixed reality environment.

2. The health exam guidance system of claim 1, wherein the one or more processors are further configured to perform operations comprising:
collecting initial data from the one or more examination devices or sensors;
applying the initial data to an initial computational model to determine a next desired maneuver for the determined type of medical examination;
continuing to collect additional data from the one or more examination devices or sensors; and
updating the initial computational model based on the additional data to produce a revised computational model to determine the next desired maneuver for the determined type of medical examination.

3. The health exam guidance system of claim 2, wherein, responsive to the collection of additional data from the one or more examination devices or sensors, the one or more processors are further configured to perform operations comprising of one or more from the group consisting of:
identifying a relevant position of an examinee; and
identifying one or more relevant landmarks of the examinee.

4. The health exam guidance system of claim 3, wherein, responsive to the collection of additional data from the one or more examination devices or sensors, the one or more processors are further configured to perform operations comprising:
assembling the additional data from the one or more examination devices or sensors, the identified position of the examinee or the identified one or more relevant landmarks of the examinee to construct a 3D examination environment; and
updating the initial computational model or the revised computational model with data describing the 3D examination environment and the identified one or more relevant landmarks to create a probability map of potential poses for the one or more examination devices or sensors.

5. The health exam guidance system of claim 4, wherein the one or more processors are further configured to:
identify, using the probability map of potential poses, one or more optimal poses, wherein the one or more optimal poses optimally satisfy a requirement of the accessed set of exam instructions.

6. The health exam guidance system of claim 5, wherein the one or more processors are further configured to perform operations comprising:
determining a pose for each of the one or more examination devices or sensors;
comparing the current pose of each of the one or more examination devices or sensors to the one or more optimal poses for each of the one or more examination devices or sensors; and
generating, in the 3D examination environment, one or more virtual items to guide a user to move each of the one or more examination devices or sensors to the one or more optimal poses for each of the one or more examination devices or sensors.

7. The health exam guidance system of claim 6, wherein at least one of one or more examination devices or sensors is an ultrasound instrument.

8. The health exam guidance system of claim 1, further comprising:
a remote data repository comprising the one or more computer storage media storing the computer readable instructions; and
a remote processing module comprising the one or more processors, wherein the one or more processors are configured to perform the operations.

9. A non-transitory computer-readable medium storing one or more instructions that, when executed by one or more processors, cause the one or more processors to perform operations comprising:
determining a type of medical examination desired;
accessing a set of exam instructions for the determined type of medical exam;
generating one or more virtual elements related to a desired maneuver indicated by the accessed set of exam instructions; and
instructing the display device to present the one or more virtual elements to the user in an augmented reality environment or in a mixed reality environment.

10. The non-transitory computer-readable medium of claim 9, wherein the stored instructions, when executed by one or more processors, cause the one or more processors to perform operations further comprising:
collecting initial data from the one or more examination devices or sensors;
applying the initial data to an initial computational model to determine a next desired maneuver for the determined type of medical examination;
continuing to collect additional data from the one or more examination devices or sensors; and
updating the initial computational model based on the additional data to produce a revised computational model to determine the next desired maneuver for the determined type of medical examination.

11. The non-transitory computer-readable medium of claim 10, wherein the stored instructions, when executed by one or more processors, cause the one or more processors to perform operations further comprising of one or more from the group consisting of:
identifying a relevant position of an examinee; and
identifying one or more relevant landmarks of the examinee.

12. The non-transitory computer-readable medium of claim 1, wherein the stored instructions, when executed by one or more processors, cause the one or more processors to perform operations further comprising:
assembling the additional data from the one or more examination devices or sensors, the identified position of the examinee or the identified one or more relevant landmarks of the examinee to construct a 3D examination environment; and
updating the initial computational model or the revised computational model with data describing the 3D examination environment and the identified one or more relevant landmarks to create a probability map of potential poses for the one or more examination devices or sensors.

13. The non-transitory computer-readable medium of claim 12, wherein the stored instructions, when executed by one or more processors, cause the one or more processors to perform operations further comprising:
identify, using the probability map of potential poses, one or more optimal poses, wherein the one or more optimal poses optimally satisfy a requirement of the accessed set of exam instructions.

14. The non-transitory computer-readable medium of claim 13, wherein the stored instructions, when executed by one or more processors, cause the one or more processors to perform operations further comprising:
determining a pose for each of the one or more examination devices or sensors;
comparing the current pose of each of the one or more examination devices or sensors to the one or more optimal poses for each of the one or more examination devices or sensors; and
generating, in the 3D examination environment, one or more virtual items to guide a user to move each of the one or more examination devices or sensors to the one or more optimal poses for each of the one or more examination devices or sensors.

15. A method, comprising:
determining a type of medical examination desired;
accessing a set of exam instructions for the determined type of medical exam;
generating one or more virtual elements related to a desired maneuver indicated by the accessed set of exam instructions; and
instructing the display device to present the one or more virtual elements to the user in an augmented reality environment or in a mixed reality environment.

16. The method of claim 15, wherein the stored instructions, when executed by one or more processors, cause the one or more processors to perform operations further comprising:
collecting initial data from the one or more examination devices or sensors;
applying the initial data to an initial computational model to determine a next desired maneuver for the determined type of medical examination;
continuing to collect additional data from the one or more examination devices or sensors; and
updating the initial computational model based on the additional data to produce a revised computational model to determine the next desired maneuver for the determined type of medical examination.

17. The method of claim 16, wherein the stored instructions, when executed by one or more processors, cause the one or more processors to perform operations further comprising of one or more from the group consisting of:
identifying a relevant position of an examinee; and
identifying one or more relevant landmarks of the examinee.

18. The method of claim 17, wherein the stored instructions, when executed by one or more processors, cause the one or more processors to perform operations further comprising:
assembling the additional data from the one or more examination devices or sensors, the identified position of the examinee or the identified one or more relevant landmarks of the examinee to construct a 3D examination environment; and
updating the initial computational model or the revised computational model with data describing the 3D examination environment and the identified one or more relevant landmarks to create a probability map of potential poses for the one or more examination devices or sensors.

19. The method of claim 18, wherein the stored instructions, when executed by one or more processors, cause the one or more processors to perform operations further comprising:
identify, using the probability map of potential poses, one or more optimal poses, wherein the one or more optimal poses optimally satisfy a requirement of the accessed set of exam instructions.

20. The method of claim 19, wherein the stored instructions, when executed by one or more processors, cause the one or more processors to perform operations further comprising:
determining a pose for each of the one or more examination devices or sensors;
comparing the current pose of each of the one or more examination devices or sensors to the one or more optimal poses for each of the one or more examination devices or sensors; and
generating, in the 3D examination environment, one or more virtual items to guide a user to move each of the one or more examination devices or sensors to the one or more optimal poses for each of the one or more examination devices or sensors.
